(19) United States
(12) Patent Application Publication
(10) Pub. No.: US 2006/0165774 A1
(43) Pub. Date: Jul. 27, 2006

(54) Non gelatin films with improved barrier properties

(76) Inventor: Edward Zbygniew Nowak, Cambridge (GB)

Correspondence Address: MCDERMOTT WILL & EMERY LLP;ATTN: INTELLECTUAL PROPERTY DEPTARTMENT, 28 STATE STREET, BOSTON, MA, 02109, US

(21) Appl. No.: 10/528,535

(22) PCT Filed: Sep. 19, 2003
(86) PCT No.: PCT/GB03/04083

§ 371 Date: Feb. 9, 2006

(30) Foreign Application Priority Data
Sep. 21, 2002 (GB) 0221986.3

(51) Int. Cl. A61K 9/48

(52) U.S. Cl. 424/451; 106/181.1

(57) ABSTRACT

Non gelatin film materials, e.g. hydroxy propyl methyl cellulose comprise e.g. an additive or additives such as an organic acid, e.g. hydroxy carboxylic acid, which form a barrier composition. The resultant films are safe human consumption and find use as a wall material of an ingestible delivery capsule, e.g. containing a dose of a pharmaceutical preparation.

### FIELD OF THE INVENTION

This invention relates to modified polymeric materials and more particularly films of the modified cellulose material hydroxy propyl methyl cellulose (HPMC), and uses of such film.

### BACKGROUND OF INVENTION

HPMC is a synthetic plastics material, which is a chemically modified form of the naturally occurring polymer, cellulose. Films, (or sheets or membranes) of HPMC are available commercially and have various uses, including proposals for use as wall materials for delivery capsules i.e. capsules designed to retain and protect their contents until an intended site of delivery or conditions of delivery are encountered, at which the contents of the capsule are released. HPMC is suitable for ingestion by humans, so delivery capsules with HPMC walls find the potential use as ingestible capsules, e.g. for the delivery of accurately metered doses of pharmaceutical preparations and dietary supplements, as a possible replacement for gelatin based capsules. See for example, WO 97/35537, WO00/27367 and WO01/03676. HPMC can be used to encapsulate substances, such as pharmaceuticals or food supplements like fish oils. It is known that certain pharmaceuticals and food supplements can be prone to attack by extended exposure to e.g. air, and It is preferable to encapsulate many unrefined vegetable oils and fish oils to prevent them from going rancid. However, even when these substances are encapsulated, e.g. within HPMC film, they can still be prone to oxidation, e.g. by the film wall of the capsule allowing oxygen present in the air outside the capsule, to pass through into the inside of the capsule and coming into contact with the capsule's contents, and reacting in some way to spoil the contents.

HPMC has poor resistance to oxygen transmission relative to other hydrocolloid film forming materials, such as gelatin, alginates, pectins and some other natural polymers. To improve oxygen barrier properties of the HPMC film, the film can be coated with hydrocolloids, for example, alginates. However, the coating of these films does give rise to certain disadvantages, such as creating films with multiple layers of materials each layer perhaps possessing different physical/chemical properties and thus creating increased processing complexities and problems arising therefrom, resulting in an increase in time and costs for film production.

Glycols and acetins are already known as film additives for certain film materials, but untreated films and films treated with acetins and/or other additives can show very poor resistance to oxygen penetration. However, it has now been surprisingly discovered that by incorporating various carboxylic acids, especially alpha hydroxy acids and beta hydroxy acids within HPMC film, it is possible to reduce the oxidation of vegetable and fish oils, and other oxidisable fill materials encapsulated in capsules made from this film.

It should be noted that this invention is not limited to simply HPMC film materials. HPC (hydroxy propyl cellulose), MHEC (methyl hydroxy ethyl cellulose), HEC (hydroxy ethyl cellulose), EHEC (ethyl hydroxy ethyl cellulose), EC (ethyl cellulose) and MC (methyl cellulose) are all materials which can be included.

### SUMMARY OF THE INVENTION

In the widest scope the of the invention, further polymeric films are contemplated, within a group which can be defined as non-gelatin polymeric films.

In one aspect of the present invention provides hydroxypropyl methyl cellulose film, comprising hydroxypropyl methyl cellulose and an additive comprising an organic acid, or derivative or salt of such an acid.

Suitable organic acids are carboxylic acids, such as mono, di, tri, or tetra or other polyvalent carboxylic acids.

Carboxylic acids according to the present invention include the following:

C1-C6 saturated or unsaturated, straight or branched chain carboxylic acids, with 1, 2, 3 or 4 carboxyl groups

C1-C6 hydroxy acids with any combination of 1, 2, 3, 4 hydroxyl/carboxyl groups, including alpha hydroxy acids (AHA's) and beta hydroxy acids (BHA's)

Cyclised acids and cyclised hydroxy acids

Specific examples of acids according to the present invention include the following:

carboxylic acids

Adipic acid

Fumaric acid

Maleic acid

Proprionic acid

Salicylic acid

Ethanoic acid

Propanoic acid

Butanoic acid

Pentanoic acid

Hexanoic acid

hydroxy acids

Alpha hydroxy butyric acid

Mandelic acid

Tartaric acid

Lactic acid

Citric acid

Malic acid

Glycolic acid

Hydroxy citric acid

cyclised acids and cyclised hydroxy acids

Gamma butyrolactone

Gamma valerolactone

Beta propriolactone

HPMC films can be treated with alpha and beta hydroxy acids and also other carboxylic acids derived from fruit acids to produce clear films which can then be used to produce capsules which can significantly reduce oxidation of certain substances encapsulated within same as compared with capsules made form HPMC treated with compounds such as glycerine, propylene glycol, poly ethylene glycol and acetins. This significant improvement in the reduction of oxidation is thought to be attributable to the acid additive incorporated within the film perhaps hindering oxygen transmission through the film.

These films can be improved or modified further to suit the application by coating these films with aqueous solutions containing the acids according to the present invention.

Therefore, in a first aspect of the invention, the one or more acids are incorporated within the film by admixing the acids within a film forming resin which is then formed into a film.

In a second aspect of the invention, aqueous solutions of the acids are applied to the surface of a preformed film.

In a third aspect of the present invention, aqueous solutions of one or more acids are applied to the surface of films which are then bonded together.

In a fourth aspect of the present invention, aqueous solutions of one or more acids are applied to the surface of one or more capsule(s) made from film according to the present invention.

Film Manufacture

HPMC is dissolved in water with an acid or acids according to the present invention e.g. citric acid, to make a solution of which the total solids being between 10-20% w/w. (During this procedure, optional ingredients such as dyestuffs, sweeteners and manufacturing aids can be added.) The resultant viscous solution is then de-aerated and extruded at a set thickness onto a moving (endless) steel belt of which, during the length of its travel is heated to 80-100 degrees centigrade. During this heating process, water is evaporated from the film, leaving a dry film of thickness between 20-150 microns. This film is then removed from the belt and is further processed for use, e.g. slitting to a final roll width, laminating the single ply film to yield a double ply film, or coating with an external coat to give a specific desired property. Alternatively, for smaller quantities of film, a viscous solution can be poured onto a flat sheet of glass, and allowed to settle to form a flat bed of viscous liquid which lies on top of the glass. This can then be introduced into an oven at the desired temperature, where it can be left to dry, to form a desired sheet of film.

Alternatively to the above, a film can be formed as above but without the inclusion of the one or more acids within the film as the film is formed. Once the film has been formed, an aqueous solution of the one or more acids is applied to the surface of the film.

Preparation of Capsules

A film solution consisting of HPMC and acid according to the present invention (total solids 10%) is cast onto glass plates to a set thickness. The cast film is then placed in a warm oven (50-80 degrees centigrade) to form a rigid film, which is then removed from the glass plate and left to equilibrate at room temperature. The resulting film produced is then placed on a vacuum forming bed and thermoformed into cavities or half capsules. Each cavity is filled (overfull) with fish or vegetable oil and lidded with an identical sheet of HPMC film. A heated tool is then used to seal the films together and to cut the resulting capsules free of excess unused film surrounding the cavities. The capsules formed are removed from the bed and packed and placed in storage.

Stability Testing

The stability of fish and vegetable oils were evaluated in the capsules made in accordance with the present invention. The stability of the oil in the capsules was evaluated by analysing the peroxide value (P.V.) over time.

Using a standard pharmaceutical test, samples were prepared and stored in HDPE bottles at 30 degrees centigrade, 60% relative humidity. Periodically, the samples were removed and analysed according to method described in the European Phamacopea: Peroxide Values Ph. Eur. method 2.5.5.

The results were plotted graphically to show comparative changes in P.V. over time.

Control capsules were made from HPMC film incorporating acetins (mono and diacetin).

The results can be interpreted thus: The higher rate of peroxide generated in the oil, the less stable is the end product.

Therefore, the best performing films show lower peroxide values.

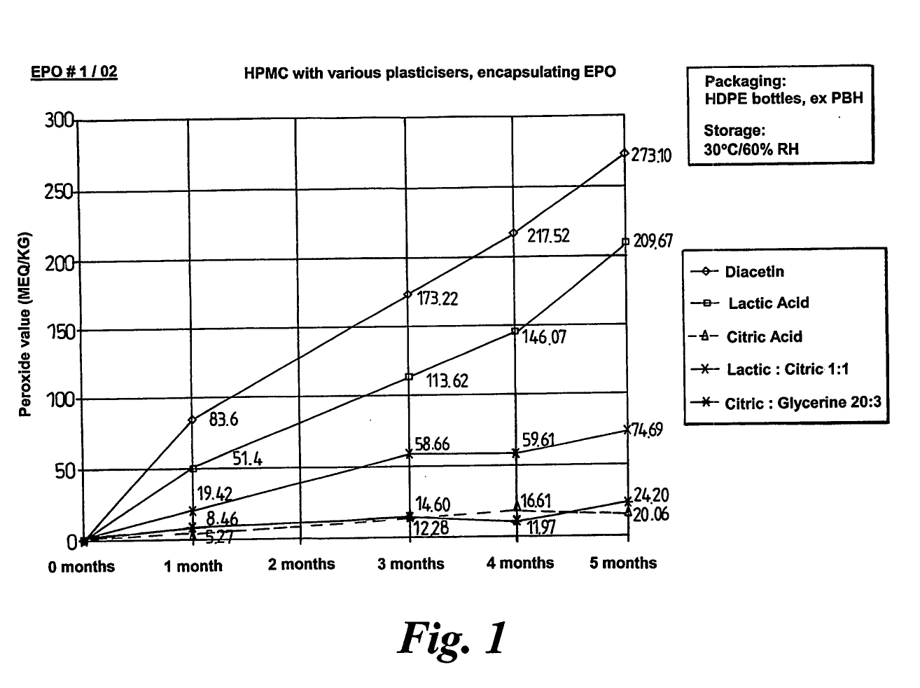

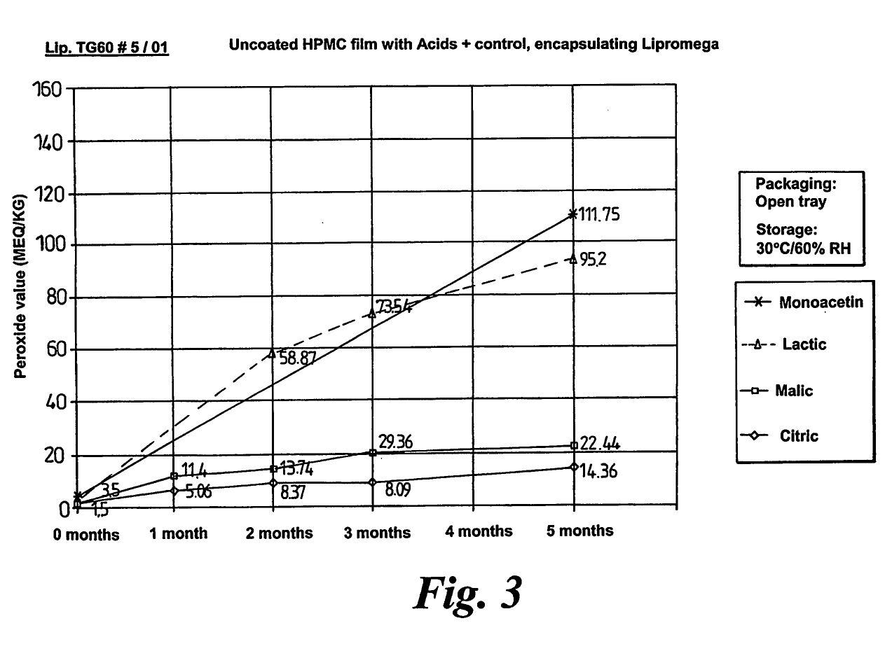

Formulations:

| | % w/w |
|---|---|
| Graph/FIG. 1, 2 and 4 | |
| HPMC (Methocel E50 ex Dow) | 77 |
| Diacetin | 23 |
| HPMC | 77 |
| Lactic acid | 23 |
| HPMC | 77 |
| Lactic acid | 11 |
| Citric acid | 12 |
| HPMC | 77 |
| Citric acid (anhydrous) | 20 |
| Glycerin | 3 |
| HPMC | 77 |
| Citric acid (anhydrous) | 23 |
| Graph/FIG. 3 | |
| HPMC | 77 |
| Monoacetin | 23 |
| HPMC | 77 |
| Lactic acid | 23 |
| HPMC | 77 |
| Malic acid | 23 |
| HPMC | 77 |
| Citric acid | 23 |

Interpretation
FIG. 1—Graph 1 Capsules Containing Evening Primrose Oil (EPO)

Demonstrates the superior performance of HPMC incorporating citric acid or citric acid/glycerin combinations within the capsule film, by revealing generally lower and slowly rising peroxide values over a 5 month period. A 1:1 lactic/citric combination in the film still demonstrates very good performance and films treated solely with lactic acid still show a marked improvement over the performance of film treated with diacetin (control), a known film additive.

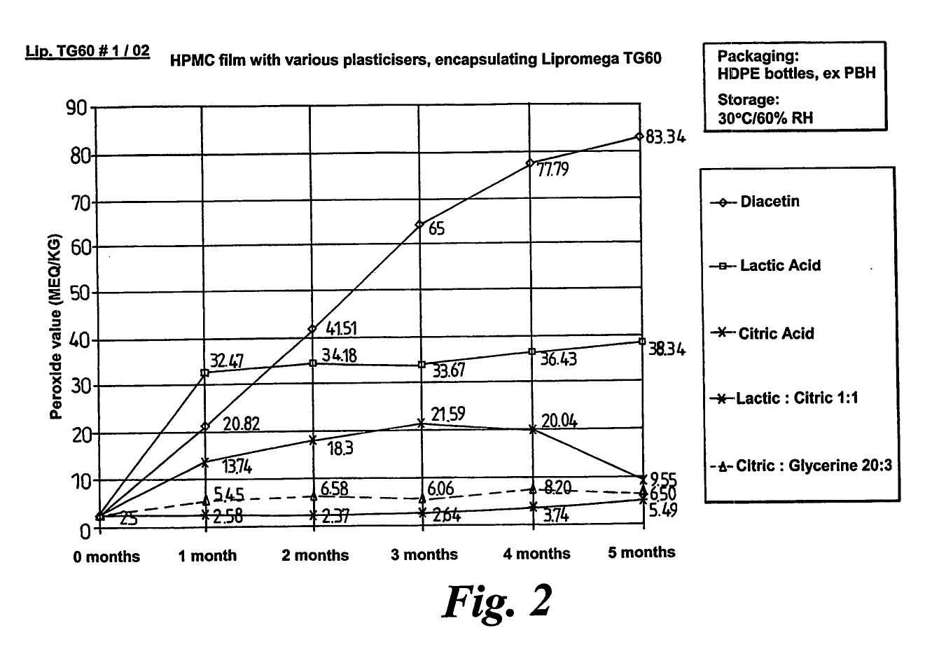

FIG. 2—Graph 2 Capsules Containing Fish Oil (Lipromega TG60)

General trends shown in graph 1 are also demonstrated here. A vast improvement in maintaining low P.V. is shown. demonstrated by the stark stablizing effect of citric acid.

FIG. 3—Graph 3 Capsules Containing Fish Oil (Lipromega TG60).

In this test, capsules were exposed directly to the atmosphere (without any packaging around the capsules). HPMC films containing citric, malic and lactic acid (especially citric and malic acids) demonstrated superior performance with respect to peroxide values, over HPMC films containing monoacetin.

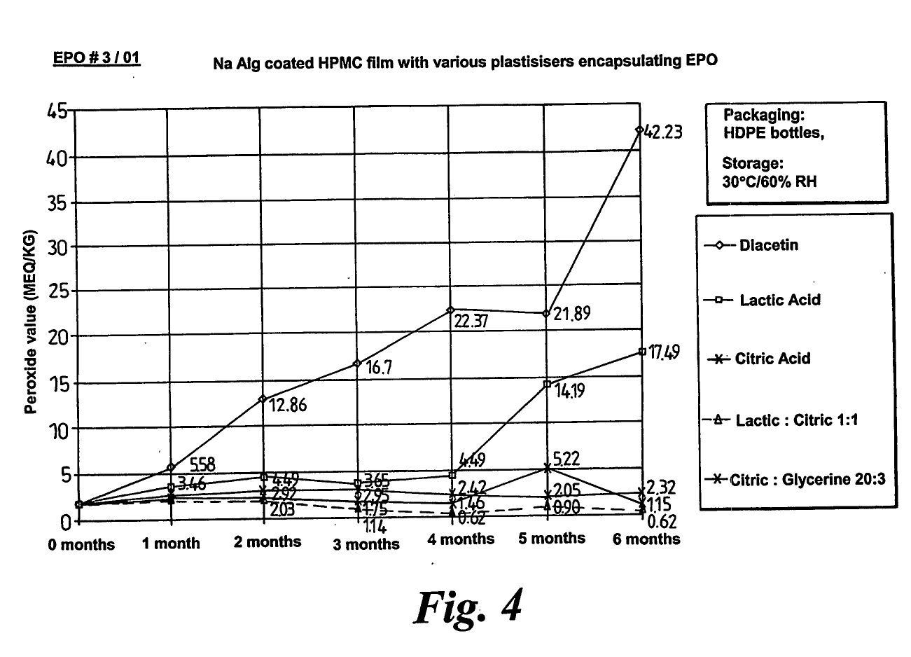

FIG. 4—Graph 4—Na Alginate Coated HPMC Film with Various Plasticisers Encapsulating EPO.

Comparing this with graph 1, this shows additional stabilization of peroxide values, which can be maintained for a longer period of time, due to the sodium alginate coating on the HPMC film.

1. A non gelatin polymeric film, comprising a non gelatin polymer and a barrier composition comprising an organic acid or a salt thereof.

2. A non gelatin polymeric film according to claim 1, wherein the non gelatin polymer is selected from the group consisting of HPMC, MHEC, HEC, EHEC, EC and MC or a combination thereof.

3. A non gelatin polymeric film according to claim 1, wherein the non gelatin polymer is HPMC.

4. HPMC film, comprising HPMC and a barrier composition comprising an organic acid or a salt thereof.

5. A non gelatin polymeric film according to claim 1, wherein the organic acid is a carboxylic acid or a salt thereof.

6. A non gelatin polymeric film according to claim 1, wherein, the organic acid is selected from the group consisting of maleic acid, fumaric acid, adipic acid, citric acid, and lactic acid.

7. A non gelatin polymeric film according to claim 1, wherein the organic acid comprises citric acid.

8. A non gelatin polymeric film according to claim 1, wherein the organic acid comprises malic acid.

9. A non gelatin polymeric film according to claims 1 or 3, wherein the organic acid is present in the amount in the range of about 5 to 40% by weight of the total weight of the film.

10. A non gelatin polymeric film according to claims 3, comprising about 23% by weight of organic acid and 77% by weight of HPMC.

11. A non gelatin polymeric film according to claim 1, wherein the film is foamed, expanded or gasified.

12. A non gelatin polymeric film according to claim 1, wherein the film has a thickness of between 20 to 250 microns.

13. A non gelatin polymeric film according to claims 1, 3, or 6 wherein the film is additionally treated with a solution comprising one or more acids selected from the group consisting of maleic acid, fumaric acid, adipic acid, citric acid, lactic acid, and salts thereof.

14. A two-ply film comprising two non gelatin polymeric films according to claim 1, wherein the two films are bonded to one another by a solution comprising one or more acids selected from the group consisting of maleic acid, fumaric acid, adipic acid, citric acid, lactic acid, and salts thereof.

15. A delivery capsule with an enclosing wall comprising a non gelatin polymeric film according to claim 1.

16. A method of producing HPMC film suitable for forming a delivery capsule, comprising the step of treating the HPMC film with an organic acid or a salt thereof.

17. A delivery capsule, whose walls provide a continuous barrier for protecting and containing the capsule's contents, said continuous barrier comprising a non-gelatin polymeric film, and an organic acid.

18. (canceled)

19. A delivery capsule as defined in claim 16, wherein the organic acid is a carboxylic acid.

20. A method of treating a non gelatin polymeric film comprising the steps of: a) making a solution of one or more organic acids; and b) applying said solution to the surface or surfaces of said non gelatin polymeric film.

21. A method of treating a non gelatin polymeric film according to claim 20, wherein said film comprises HPMC.

22. A method of treating a non gelatin polymeric film according to claim 21, wherein said solution of one or more organic acids comprises one or more carboxylic acids.

23. A delivery capsule having walls, wherein said walls have adsorbed or absorbed, from the outer side of the walls, a barrier solution comprising one or more carboxylic acids.

24. A delivery capsule having walls, wherein said walls comprise an outerpart and an innerpart, and further wherein said walls have a gradation in concentration of one or more carboxylic acids, through the thickness of the walls.

25. A delivery capsule according to claim 24, wherein the outerpart of the walls possesses the most concentration of one or more carboxylic acids and the inner part of the walls possesses the most concentration of one or more carboxylic acids.

26. A delivery capsule according to claim 24, wherein the inner part of the walls possesses the most concentration of one or more carboxylic acids and the outer part of the walls possesses the least concentration of one or more carboxylic acids.
